(19) United States
(12) Patent Application Publication
(10) Pub. No.: US 2011/0178382 A1
(43) Pub. Date: Jul. 21, 2011

(54) BLOOD GLUCOSE IGNITION INTERLOCK

(76) Inventor: Claire H. Topp, Eden Prairie, MN (US)

(21) Appl. No.: 12/689,907

(22) Filed: Jan. 19, 2010

(51) Int. Cl. A61B 5/145

(52) U.S. Cl. 600/365

(57) ABSTRACT

The present invention relates to an ignition interlock that requires a blood glucose level measurement prior to permitting ignition of a vehicle. The interlock prevents operation of equipment in which it is installed if a blood glucose level measurement is not taken or if the resultant measurement is outside of a desired range. In another embodiment, a method for ignition interlock is provided. The method includes activating an identification system and activating an ignition interlock system based on information from the identification system. The method further comprises testing a blood glucose level of the user to generate a determined blood glucose level and comparing the determined blood glucose level to preset blood glucose levels, wherein the preset blood glucose levels include an acceptable minimum blood glucose level. Ignition is enabled or prevented based on the comparison. More particularly, ignition is enabled if the determined blood glucose level is above the acceptable minimum blood glucose level and ignition is prevented if the determined blood glucose level is below the acceptable minimum glucose level.

### FIELD OF THE INVENTION

The present invention relates to an ignition interlock that requires a blood glucose level measurement prior to permitting ignition of a vehicle.

### BACKGROUND OF THE INVENTION

Ignition interlocks are currently available for the prevention of drunk driving. More specifically, to prevent convicted drunk drivers from continuing to drive while intoxicated, ignition interlocks may be installed in convicted drivers' vehicles to prevent the vehicles from being started while the driver is intoxicated. Known interlock devices connect breath-alcohol analyzers to a vehicle's ignition system.

With typical ignition interlock devices, a driver must pass a breath test by blowing into the device before starting the vehicle. In other ignition interlock devices, a light is directed onto the drivers finger and the blood alcohol level is determined by assessment of the light reflected or passing through the finger. Regardless of manner of measuring blood alcohol level, unless the driver passes the test, the vehicle will not start. Some units also conduct tests at random intervals once the vehicle has been started to deter the driver from drinking after the engine has been started and from continuing to drive while intoxicated.

Someone driving while hypoglycemic (i.e., having low blood sugar) can present very similarly to someone driving while intoxicated. As a result, there is a relatively high frequency of unjustified DUI or DWI arrests stemming from hypoglycemic symptoms that can closely mimic those of a drunk driver. More seriously, there is also a relatively high frequency of car accidents stemming from hypoglycemic events (i.e., abnormally low levels of blood glucose/blood sugar). A study from the University of Virginia Health System reports that drivers with type 1 diabetes may be more prone to car crashes than non-diabetics. The study found that over 12 months, 52% of the 452 drivers in the study reported at least one hypoglycemia-related driving mishap and 5% reported six or more. Driving Mishaps Among Individuals With Type 1 Diabetes: A Prospective Study. Diabetes Care, Volume 32, No. 12, December 2009. According to the lead author of the study who has authored several studies on the same subject, Daniel J. Cox: “Type 1 diabetic drivers reported significantly more crashes, moving violations, episodes of hypoglycemic stupor [caused by extremely low blood sugar], and required assistance while driving compared to type 2 diabetics or spouse-control subjects.” Diabetes and Driving Mishaps—Frequency and correlations from a multinational survey, Diabetes Care, Volume 26, No. 8, August 2003.

Type 1 diabetes develops when the pancreas can no longer manufacture insulin, the hormone necessary to move sugar out of the blood and into tissues where it supplies energy. A type 1 diabetic must rely on injected insulin (whether provided through injection shots or an insulin pump) for this purpose. The body, using insulin, breaks down the natural sugars (carbohydrates) that are eaten and converts them to glucose, which can then be absorbed from the intestines into the blood. At least because of the manual correlation of carbohydrates eaten to insulin required, there is the potential for blood sugar levels to either rise too high or drop too low. A drop in blood sugar may precipitate dangerous driving.

In an individual with normal metabolism, blood glucose levels are regulated precisely and kept within a narrow range (typically between approximately 70 mg/dl and 110 mg/dl). Diabetics (and individuals who have certain kinds of tumors or possess other errors of metabolism) are unable to naturally regulate their glucose levels, which can range from extremely high glucose levels or to dangerous low glucose levels. By laboratory definition, hypoglycemia is defined as a blood sugar level below 60 mg/dl. Symptoms of hypoglycemia are usually divided into those affecting the body and those affecting the brain or central nervous system (CNS). Bodily symptoms may include rapid heartbeat, sweating, tremors, anxiety, hunger, and nausea. Those affecting the CNS are light-headedness, confusion, headache, loss of consciousness, seizures, delayed reflexes, and slurred speech. All of these symptoms (affecting body and affecting CNS) are directly related to the brain or body not receiving enough glucose for use as an energy source.

While in a hypoglycemic state, a person is often unaware of the hypoglycemic state or unaware of the severity of the hypoglycemic state. More particularly, someone in a hypoglycemic state often will not be aware of how serious their symptoms are while engaged in activities such as driving, which require a high degree of concentration. This lack of awareness makes it difficult for diabetics to know that they need test their blood sugar before driving—at a time when they need to test their blood sugar levels the most. It is also possible for blood sugar to plunge quite suddenly, causing the individual to lose consciousness completely. This is such a serious problem for those with endocrine problems that numerous studies of hypoglycemia's impact on driving ability and other activities that require a high degree of concentration have been carried out and reported on extensively in the medical literature.

“There's always the concern that patients who experience low blood sugar—hypoglycemia—may be at greater risk when driving or performing other tasks that require mental concentration and high performance,” says Dr. Kenneth Hupart, chief of endocrinology, metabolism and diabetes at Nassau University Medical Center in East Meadow, N.Y. “If you're driving during a period of hypoglycemia, for example, your reaction time and judgment might be impaired,” Hupart explains.

Because diabetes is a medical condition, many states react to a single hypoglycemic driving incident by suspending or revoking a diabetic's license. Such reaction significantly impairs a diabetic's ability to work and simply to do normal daily activities. Further, because, unlike alcohol consumption where a drunk driver is construed as having made a choice in driving drunk, a diabetic who becomes hypoglycemic while driving, did not make a choice to do so. Short of having a mechanism that requires a diabetic driver to test his or her blood sugar before starting a car, there is little a diabetic can do to assure the state that such incidents will not happen in the future—given the likelihood that the diabetic had no desire for it to happen in the first place.

Thus, a need exists for an ignition interlock that requires a blood glucose level measurement prior to permitting ignition of a vehicle.

### SUMMARY OF THE INVENTION

The present invention relates to an ignition interlock that requires a blood glucose level measurement prior to permitting ignition of a vehicle. The interlock prevents operation of equipment in which it is installed if a blood glucose level measurement is not taken or if the resultant measurement is outside of a desired range. While ignition interlock devices are known for analyzing blood alcohol, none are available for blood glucose monitoring.

In one embodiment, an ignition interlock system for a vehicle is provided comprising a glucometer, a microprocessor, an ignition circuit, and an annunciator panel. The glucometer measures blood glucose levels to develop a determined blood glucose level. The microprocessor is operably coupled to the glucometer and is configured for comparing the determined blood glucose level measured by the glucometer to preset blood glucose levels and for transmitting a signal based on the comparison. In an open condition the ignition circuit prevents ignition of the vehicle. In a closed condition, the ignition circuit enables ignition of the vehicle. The relay is configured for processing the signal from the microprocessor to the ignition circuit and the signal controls opening or closing of the ignition circuit. The annunciator panel includes lights for indicating status of the ignition interlock system.

In another embodiment, a method for ignition interlock is provided. The method includes activating an identification system and activating an ignition interlock system based on information from the identification system. The method further comprises testing a blood glucose level of the user to generate a determined blood glucose level and comparing the determined blood glucose level to preset blood glucose levels, wherein the preset blood glucose levels include an acceptable minimum blood glucose level. Ignition is enabled or prevented based on the comparison. More particularly, ignition is enabled if the determined blood glucose level is above the acceptable minimum blood glucose level and ignition is prevented if the determined blood glucose level is below the acceptable minimum glucose level.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed description is to be regarded as illustrative in nature and not restrictive.

### BRIEF DESCRIPTION OF THE DRAWINGS

FIG. 1 illustrates a schematic view of a an ignition interlock system as provided in a vehicle in accordance with one embodiment.

FIG. 2 illustrates a block diagram of a method of using an ignition interlock in accordance with one embodiment.

FIG. 3 illustrates a schematic representation of an ignition interlock system in accordance with one embodiment.

While the subject invention will now be described in detail with reference to the figures, and in connection with the illustrative embodiments, changes and modifications can be made to the described embodiments without departing from the true scope and spirit of the present invention as defined by the appended claims.

### DETAILED DESCRIPTION OF THE PREFERRED EMBODIMENTS

The present invention relates to an ignition interlock that requires a blood glucose level measurement prior to permitting ignition of a vehicle. The interlock prevents operation of equipment in which it is installed if a blood glucose level measurement is not taken or if the resultant measurement is outside of a desired range.

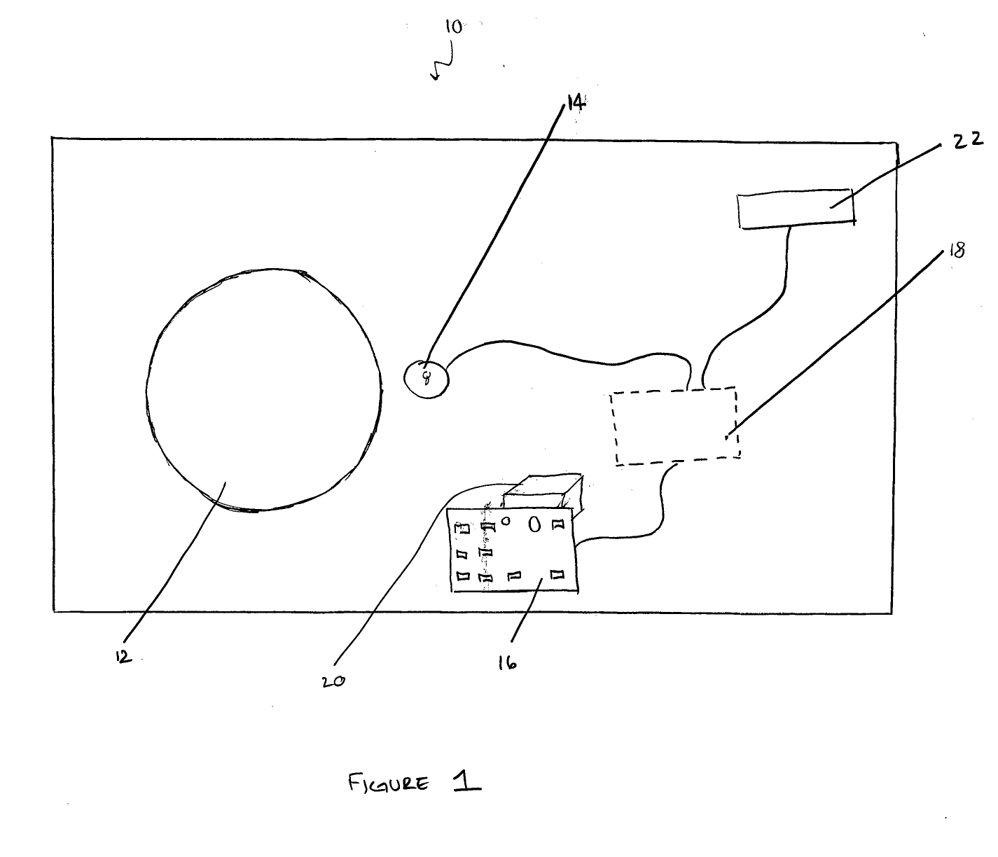

As shown in FIG. 1, a vehicle may be provided with an ignition interlock system 10. The vehicle includes a steering wheel 12, an ignition center (such as a port for receiving an ignition key) 14, a panel 16, and a microprocessor 18. A glucometer 20 is provided associated with the panel 16 and microprocessor 18. The ignition interlock system may further include an identification system. The vehicle includes an ignition system linked to the microprocessor of the ignition interlock system. In some embodiments, the ignition interlock system may be linked to an emergency call system, such as via OnStar. Accordingly, the vehicle may include an emergency call apparatus 22 operably associated with the microprocessor.

The relative arrangement of the ignition interlock system to the vehicle ignition system and the vehicle operating system, including for example the steering wheel, may vary. The exact arrangement may depend on, among other things, the type of glucometer. In some embodiments, the ignition interlock glucometer may be provided within a detection unit housing the glucometer and, optionally, an identification system.

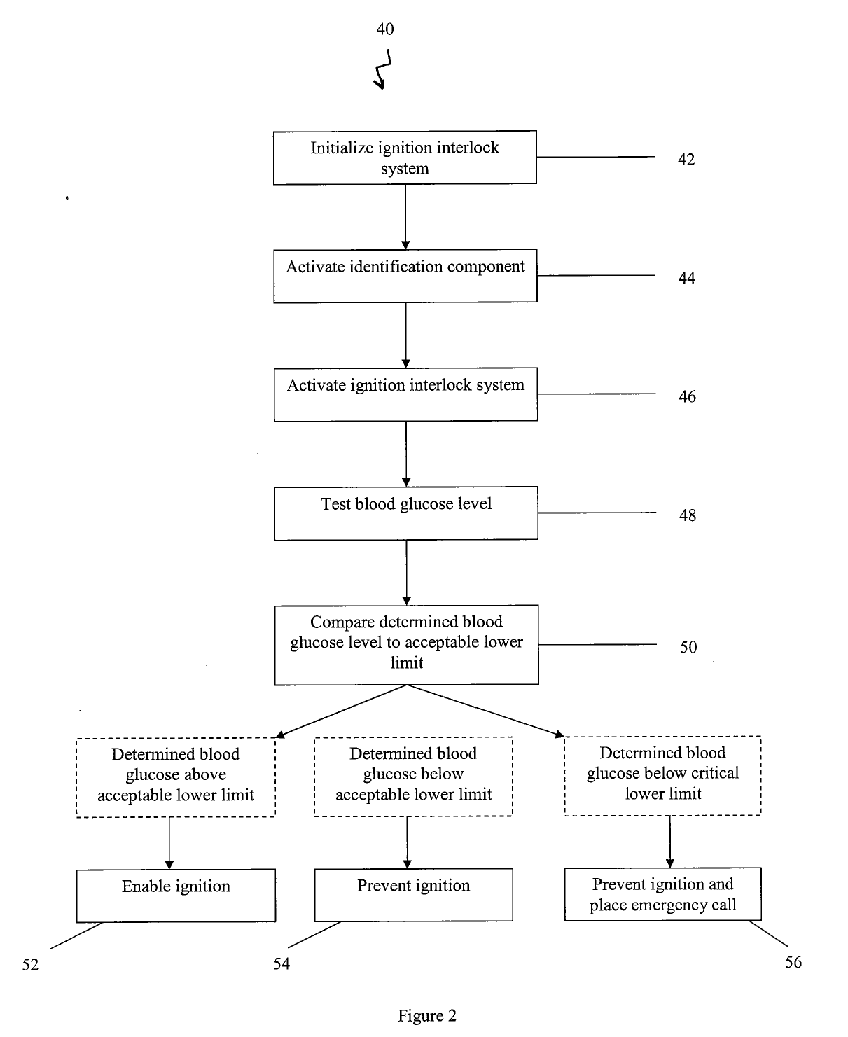

FIG. 2 illustrates a block diagram of a method 40 of using an ignition interlock as provided herein. Prior to normal use of the system, and as described more fully below, the system may be initialized [block 42.] To initialize the system, the diabetic inputs an acceptable lower limit for blood glucose levels for that person into the ignition interlock system. In contrast to, for example, alcohol levels where a static number is the legal limit for all individuals (at least within a certain jurisdiction), the acceptable low glucose level for one diabetic can vary from the acceptable low glucose level for another diabetic. Other information regarding blood glucose levels may be input by the diabetic such as a critical lower limit or a range of acceptable lower limits. Generally, the system may only be initialized a single time. However, in some situations, it may be desirable to re-initialize the system, such as when the diabetic's treatment protocol is changed.

In some embodiments, the ignition interlock system may have an identification component such that only the diabetic for whom the ignition interlock is provided is required to go through ignition interlock testing prior to starting the vehicle. This would allow persons other than the diabetic to use the car without having to test their blood sugar. Any suitable identification component may be used including, for example, fingerprint identification, retinal identification, voice identification, blood identification, special key or other. In some embodiments, identification information may be input at initialization [block 42].

After the system has been initialized, the ignition interlock is activated [block 46] when a user seeks to start a car (or other vehicle). The system may be activated in any suitable manner for indicating desired starting of the car. For example, this may comprise inserting a key into the ignition, pressing a button, or other. Once the ignition interlock system is activated, it must be passed or bypassed before the vehicle may be started. Passing generally is intended to mean detection of an acceptable blood glucose level. Bypassing generally is intended to mean any mechanism by which the ignition interlock system may be deliberately skipped, such as via an identification component that identifies that the driver is not the diabetic.

If provided, the identification component may be activated [block 44] when the ignition interlock system is activated [block 46]. It is to be appreciated that, in some embodiments, no identification component may be provided such that any driver using the vehicle may be requested to undergo blood glucose testing. Further, in some embodiments, the diabetic may choose not to activate or use the identification system and may instead proceed directly to blood glucose testing.

In some embodiments, the identification component may bypass the ignition interlock system, such as when the user is identified as not the diabetic. For example, a first ignition key may be provided to a first user (the diabetic) and a second ignition key may be provided to a second user (the non-diabetic). When the first key is inserted in the ignition, the ignition interlock system may be activated. When the second key is inserted in the ignition, the ignition interlock system may be bypassed. Thus, if the user is not the diabetic, the system releases the lock on ignition and the user can start the vehicle.

If no identification is given that the driver is not the diabetic (for example by designated ignition key, positive identification of a non-diabetic, or merely no usage of an identification system), the ignition interlock system requires passing of the a glucose reading test before permitting starting of the vehicle. If the user is the diabetic, the system requires the user to pass the ignition interlock testing before allowing the user to start the car.

Upon activation of the ignition interlock [block 46], the user is prompted to use a blood glucose monitor to determine blood glucose level. As may be appreciated by one skilled in the art, any suitable glucometer method or device may be used to determine blood glucose level. Accordingly, the user tests their blood glucose level [block 48]. The result of this test is a “determined blood glucose level”. The determined blood glucose level is compared to the acceptable lower limit that is input into the system [block 50]. If the determined blood glucose level exceeds the acceptable lower limit, ignition is enabled [block 52]. If the determined blood glucose level is below the acceptable lower limit, ignition is prevented [block 54]. In some embodiments, if the determined blood glucose level is below the critical lower limit, an emergency call may be placed [block 56].

Even after ignition of the vehicle is permitted, the diabetic may be required to undergo further blood glucose testing at time intervals after ignition. Requirement of a subsequent blood glucose test may be contrasted with systems using alcohol interlocks where it is unlikely that a user's blood alcohol level will rise substantially after passing the interlock testing (unless the user is intentionally trying to avoid the system). Further, physiology naturally tends to result in blood alcohol levels lowering as time passes. This is considered good with respect to the alcohol ignition interlock systems. In contrast, rise or fall of blood glucose levels is not predictable purely on the basis of food intake (or lack thereof) because blood glucose depend on food intake, insulin administered, and activity levels. For example, if too much insulin was taken (or not enough food was eaten), blood glucose levels will continue to drop until more sugar is consumed. Thus, a period of time may be set, for example approximately two (2) hours, after which the user is required to repeat a blood glucose test before the car will continue. In other embodiments, if the determined blood glucose level is in the acceptable lower limit range, such that the blood glucose level may be nearing the acceptable lower limit, the user may be required to do a further blood glucose test within a specified time period such as thirty (30) minutes. In some embodiments, warning notices may be given that a further blood glucose test is due on a periodic basis before it is due. The system may be configured such that, when the blood glucose test is due, the car becomes inoperable, for example, by shutting down or going into neutral after adequate warning to the driver.

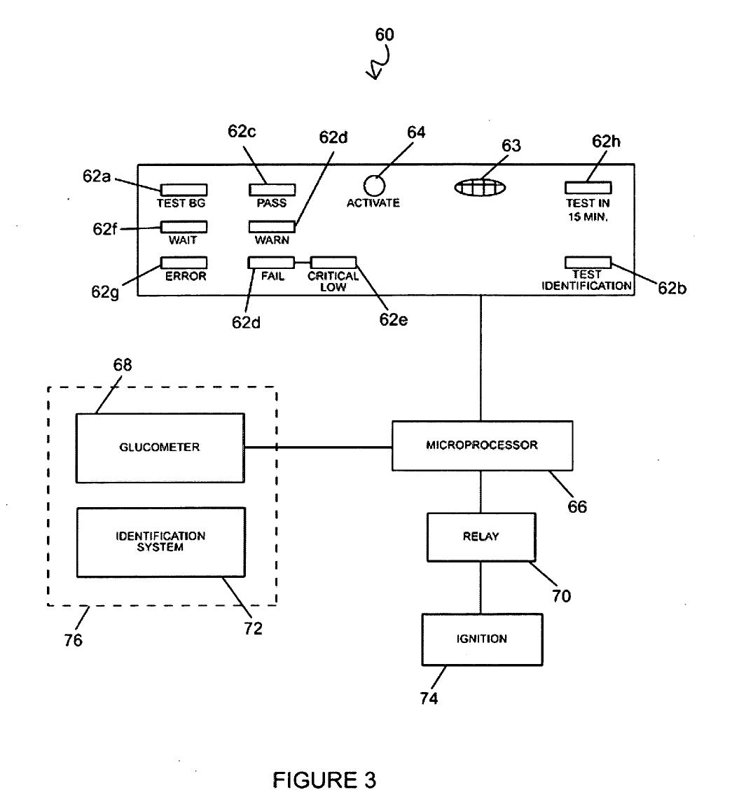

One embodiment of a suitable ignition interlock system is shown in FIG. 3. An annunciator panel 60 is provided having a plurality of lights 62a-62h that indicate the status of the interlock. The panel 60 may be provided with an activation mechanism 64 for activating the system. In the embodiment shown, the activation mechanism 64 is a button that may be depressed by the user. As previously described, the system may alternatively be activated when an ignition key is put into the ignition or a user otherwise seeks to start the car.

The annunciator panel 60 is electronically connected to a microprocessor 66 which electronically controls the functioning of the interlock. The microprocessor 66 may be provided with an information recording and storage medium, such as a magnetic cassette or disk in an appropriate drive, or other electronic volatile or nonvolatile memory. The microprocessor 66 is further linked to a glucometer 68, a relay 70, and, optionally, an identification system 72.

The relay 70 is operably connected to the ignition 74 and is switchable by the microprocessor 68 to open or close an ignition circuit of the ignition 74 in the vehicle. The ignition circuit supplies electrical power necessary for the vehicle to be operated. Thus, the vehicle may only be started when the ignition circuit is closed. When the circuit is open, the equipment does not receive its electrical current needed for it to run. The ignition circuit may supply power to mechanisms such as starters or spark plugs, as is known to those skilled in the art.

As shown, an identification system 72 may be provided linked to the microprocessor. The identification system 72 may be, for example, a fingerprint panel. Thus, upon activation by a user, the user may be prompted to put his finger on the panel to provide a fingerprint to the system. The fingerprint is transmitted to the microprocessor 66. The microprocessor 66 compares the fingerprint to the fingerprint information that was previously input during initialization of the ignition interlock system (block 52 of FIG. 2). In embodiments where the identification system comprises a fingerprint system, the microprocessor may be provided with a comparator that compares the fingerprint image with the. prestored image of the diabetic's fingerprint and determines whether the images match, thus ascertaining whether the analyzed finger belongs to the diabetic. In alternative embodiments, other identification systems may be provided. In yet other embodiments, no identification systems may be provided.

If, based on identification information input (such as a fingerprint) to the identification system 72, the microprocessor 66 determines that the user is not the diabetic for whom the ignition interlock was provided, the ignition interlock is bypassed and ignition circuit is closed. If the microprocessor 66 determines that the user is the diabetic, the circuit remains open and glucose testing is required.

A glucometer 68 is linked to the microprocessor 66. The microprocessor 66 controls and processes readings from the glucometer 68. Any suitable glucometer 68 may be used. Most commonly, glucometers require a test strip and for blood to be applied to the test strip. Accordingly, the invention is described with a blood testing glucometer for the purposes of illustration. Alternatively, the glucometer 68 may be a glucometer that operates by applying light to a tissue and reading analytes in the tissue based on the applied light. In yet other embodiments, the ignition interlock may be tied to a continuous glucose monitor system used by the user, such as a glucose level watch (such as the GlucoWatch, available from Cygnus Inc.) or a semi-implanted continuous glucose monitor (such as the Guardian, available from Medtronic). In some embodiments, the glucometer 68 may be provided by the diabetic and tied to the ignition interlock system such that any commercially available glucometer may be used.

As previously discussed, the glucometer 68 may be provided within a detection unit 76 housing the glucometer and, optionally, an identification system 72. The detection unit 76 may include a housing and a port, the port being suitable sized for accommodating a test strip. Such test strip may be inserted through the hole from the user or may be supplied from within the detection unit to the driver, depending on the type of glucometer used. Many currently available test strips are sensitive to temperature variations and may become corrupt if left in an environment with extreme temperatures. Thus, in one embodiment, the user supplies the test strips and inserts a test strip through the port into the glucometer for ignition interlock testing. The port is diagrammatically shown at 63 on annunciator panel 60. It is to be appreciated that the port may be provided in alternative locations in the vehicle, as discussed with respect to FIG. 1.

The glucometer 68 transmits the measured glucose level to the microprocessor 66. The microprocessor then compares the measured glucose level with information previously input when the ignition interlock was initialized (block 52 of FIG. 2), such as a lower acceptable level, a lower acceptable range, and a critical lower level.

As previously described, before the interlock system is utilized, acceptable glucose levels may be entered into the microprocessor. Acceptable ranges of glucose levels are different for different diabetics. While 70-110 mg/dl is generally the normal range for a non-diabetic, some diabetics begin feeling symptoms of hypoglycemia at 100 mg/dl, while others have no symptoms before 50 mg/dl. Various mechanisms for locking the set acceptable glucose levels may be used. Thus, for example, the microprocessor may be removable such that a doctor, state agency, or other person not intended to operate the vehicle may input the acceptable lower blood glucose limit. Alternatively, the microprocessor may be configured for remote communication such that information input at, for example, a computer is transmitted to the microprocessor. After the acceptable lower blood glucose limit has been entered, this level may be locked into the microprocessor in a manner such that it cannot be modified.

If the microprocessor determines that the measured glucose level exceeds the lower acceptable level, the ignition circuit is closed. If the microprocessor determines that the measured glucose level is below the lower acceptable level, the ignition circuit is left open. If the microprocessor determines that the measured glucose level is at or less than the critical lower limit, the emergency call system may be activated and a preset number dialed (for example 911 or an emergency contact).

Accordingly, in one embodiment, when an operator wishes to start the vehicle, he or she actuates that activation switch 64 on the annunciator panel 60, energizing the interlock system. The system remains energized until a preselected time period has elapsed or the user has undergone blood glucose testing. When the energized system is ready to begin testing, a test light on the annunciator panel illuminates. In various embodiments, the test light may be a blood glucose test light (also referred to as a glucose level test light) 62a or may be an identification test light 62b.

In some embodiments, the first illuminated test light is an identification test light 62b. Thus, when an identification test light 62b is illuminated, the user is prompted to use the identification system 72.

Upon determining that the user is the diabetic, a glucose level test light 62a is illuminated. The user is thus prompted to use the glucometer 68. After input of data to the glucometer 68, such as by deposit of blood on a test strip, but before processing of the result by the microprocessor, a wait light 62f may be illuminated. The microprocessor 66 uses data from the glucometer 68 to determine that the test is passed if the determined glucose level is above the acceptable lower limit. If the test is passed, the microprocessor causes a pass light 62c on the annunciator panel to illuminate and activates the relay 70 to the ignition 74 that closes the ignition circuit, allowing the vehicle to be started and operated. In some embodiments, the microprocessor 66 may record in its memory the glucometer test results. If determined blood glucose level was above the acceptable lower limit but within the acceptable lower range, a warn light 62d may be illuminated instead of, or in addition to, the pass light 62c.

During operation of the vehicle, a test in time interval (for example 15 minutes) light 62h may be illuminated. The test in time interval 62h light may be set to automatically be illuminated after a time interval of operation, such as after 2 hours of continuous driving, and/or may be set to be illuminated after a time interval of operation after a test result in the acceptable lower range, such as 30 minutes. The test in time interval 62h provides the user of warning that operation of the vehicle may be prevented in that time interval to force testing of blood sugar.

If the test is failed because the determined blood glucose level is lower than the acceptable lower limit, the microprocessor illuminate a fail light 62d on the annunciator panel. If the test is failed because the determined blood glucose level is below the critical lower limit, the microprocessor illuminates a critical low light 62e and activate an emergency call. If a test is failed, the interlock prevents the vehicle from being operated.

While the invention is described primarily in reference to preventing ignition prior to operation of the vehicle, it is to be appreciated that the ignition interlock system may be used after operation of the vehicle has started, for example when the system first tests a passing but lower blood glucose level and requires a further test after a time interval has elapsed. In such situation, a different manner of preventing operation may be used given that ignition may have already been permitted. Thus, manners in which vehicle operation can be prevented include preventing it from starting, preventing it from running, or by creating a nuisance to an operator's continued running of the equipment. Such a nuisance can be generated by honking a loud horn on the equipment, or by flashing highly visible lights.

If the test did not provide a usable result, such as because not enough blood was deposited on the test strip, because the temperature was not suitable for testing blood glucose, or other, an error light 62g may be illuminated. When the error light 62g is illuminated, ignition remains prevented.

A “wait” light may be illuminated when the microprocessor is preparing for a test, such as when the system is being reset between tests.

What is claimed is:

1. An ignition interlock system for a vehicle comprising:
a glucometer for measuring blood glucose levels to develop a determined blood glucose level;
a microprocessor operably coupled to the glucometer, the microprocessor being configured for comparing the determined blood glucose level measured by the glucometer to preset blood glucose levels and for transmitting a signal based on the comparison;
an ignition circuit wherein, in an open condition, ignition of the vehicle is prevented and, in a closed condition, ignition of the vehicle is enabled;
a relay configured for processing the signal from the microprocessor to the ignition circuit and wherein the signal controls opening or closing of the ignition circuit; and
an annunciator panel including lights for indicating status of the ignition interlock system.

2. The ignition interlock system of claim 1, further comprising an identification system.

3. The ignition interlock system of claim 2, wherein the identification system comprises a fingerprint identification system.

4. The ignition interlock system of claim 1, wherein the identification system and the glucometer are housed in a detection unit.

5. The ignition interlock system of claim 3, wherein the detection unit includes a port for accommodating a glucose test strip.

6. The ignition interlock system of claim 1, further comprising an emergency call system for performing an emergency call if the determined blood glucose level is below a preset critical low level.

7. A method for ignition interlock comprising:
activating an identification system;
activating an ignition interlock system based on information from the identification system;
testing a blood glucose level of the user to generate a determined blood glucose level;
comparing the determined blood glucose level to preset blood glucose levels, wherein the preset blood glucose levels include an acceptable minimum blood glucose level;
enabling or preventing ignition based on the comparison, wherein ignition is enabled if the determined blood glucose level is above the acceptable minimum blood glucose level and ignition is prevented if the determined blood glucose level is below the acceptable minimum glucose level.

8. The method of claim 7, wherein the preset blood glucose levels include a critical low blood glucose level.

9. The method of claim 8, further comprising activating an emergency call if the determined blood glucose level is at or below the critical low blood glucose level.

10. The method of claim 7, wherein the preset blood glucose levels include an acceptable low blood glucose range.

11. The method of claim 10, further comprising requiring a further blood glucose test after a predetermined time period when the determined blood glucose level is within the acceptable low blood glucose range.

12. The method of claim 7, further comprising requiring a further blood glucose test after a predetermined time period of continuous driving.

13. The method of claim 7, further comprising initializing the ignition interlock system prior to use of the ignition interlock system.

14. The method of claim 13, wherein initializing the ignition interlock system comprises inputting the acceptable low blood glucose level.

15. The method of claim 14, wherein inputting an acceptable low blood glucose level is done remotely.

16. The method of claim 14, wherein initializing the ignition interlock system further comprises inputting an acceptable low blood glucose range and a critical low blood glucose level.

17. The method of claim 13, wherein initializing the ignition interlock system comprises inputting identification information identifying a diabetic user.

18. An ignition interlock system for a vehicle comprising:
a microprocessor operably coupled to a glucometer, the microprocessor being configured for comparing a determined blood glucose level measured by the glucometer to preset blood glucose levels and for transmitting a signal based on the comparison;
an ignition circuit wherein, in an open condition, ignition of the vehicle is prevented and, in a closed condition, ignition of the vehicle is enabled; and
a relay configured for processing the signal from the microprocessor to the ignition circuit based.
